(19) United States
(12) Patent Application Publication
(10) Pub. No.: US 2008/0003136 A1
(43) Pub. Date: Jan. 3, 2008

(54) Automated disinfectant station

(76) Inventors: Rod White, Nashville, AR (US); Scott E. Pace, Nashville, AR (US)

Correspondence Address: John E. Vandigriff, 906 Stone Circle Ln, Lewisville, TX, 75056, US

(21) Appl. No.: 11/477,116

(22) Filed: Jun. 29, 2006

(51) Int. Cl. A61L 2/00

(52) U.S. Cl. 422/62; 422/292

(57) ABSTRACT

A disinfectant station disinfects vehicles as they pass through an automated station. As the vehicle approaches the station, spray units are turned on and water and a disinfectant are mixed to spray the wheels and under side of the vehicle. The system is turned on by a motion detector, and if it is at night, a light is also turned on. After the vehicle has passed through the disinfectant station, the spray is turned off. The motion detector turns on both the spray stations and light. If it is daytime, then the lights are not turned on. The spray stations are low pressure to adequately spray disinfectant on the vehicle, but does not clean or remove dirt or other material from the vehicle. The low pressure spray conserves the amount of disinfectant sprayed, spraying only that amount need to disinfect the vehicle. Sprayers are placed to spray the outside and inside faces of the wheels and the sides and under side of the vehicle.

### FIELD OF THE INVENTION

The invention relates to a disinfectant station, and more particularly to a disinfectant station for automatically spraying vehicles as they move through the disinfectant station.

### BACKGROUND OF THE INVENTION

The prior art has may patents related to the cleaning of vehicles, the tires of vehicles and the under carriages of vehicles. The following listed patent fall into one of the above categories, but does not disclose a system for disinfecting a vehicle. U.S. Pat. Nos. 4,979,536, 5,161,557, 5,463,788, 6,358,330, 6,992,595 and Des 394,921.

The listed patents are for the purpose of showing possible related prior art and are not detrimental to or show or suggest the present invention.

### SUMMARY OF THE INVENTION

The invention is a disinfectant station that disinfects vehicles as they pass through an automated station. As the vehicle approaches the station, spray units are turned on and water and a disinfectant are mixed to spray the wheels, sides and under side of the vehicle. The system is turned on by a motion detector, and if it is at night, a light is also turned on. After the vehicle has passed through the disinfectant station, the spray is turned off. The motion detector turns on both the spray stations and light. If it is daytime, then the lights are not turned on. The spray stations are low pressure to adequately spray disinfectant on the vehicle, but does not clean or remove dirt or other material from the vehicle. The low pressure spray conserves the amount of disinfectant sprayed, spraying only that amount need to disinfect the vehicle. Sprayers are placed to spray the outside and inside faces of the wheels and the sides, and under side of the vehicle.

The technical advance represented by the invention as well as the objects thereof will become apparent from the following description of a preferred embodiment of the invention when considered in conjunction with the accompanying drawings, and the novel features set forth in the appended claims.

### BRIEF DESCRIPTION OF THE DRAWINGS

FIG. 1 shows the disinfectant station controls;

FIG. 2 shows a drive through spray station; and

FIG. 3 shows the drive through spray station of FIG. 2 with addition sprayers.

### DESCRIPTION OF A PREFERRED EMBODIMENT

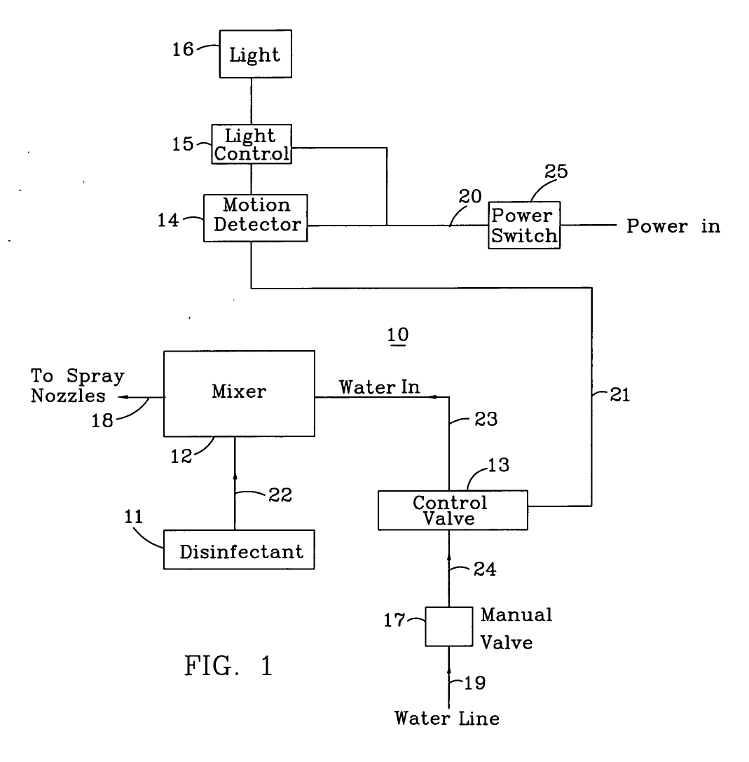

FIG. 1 shows a diagram of an example of a disinfectant station control. The disinfectant station may be use, for example, at poultry farms where it is desirable not to introduce into or take out diseases such as the bird flu. The disinfectant station will disinfectant incoming and out going vehicles. In this manner, viruses and diseases are disinfected from the incoming and out going vehicles, preventing the spread of the diseases. While not limited to a particular disinfectant, a disinfectant such as one named Agriquat may be used.

In the disinfectant station 10 in FIG. 1, a mixer receives a disinfectant 11 through line 22 and water though line 23, mixes them, and directs them through line 18 to spray nozzles. Water is received from a water line 19 and flows through a manual ON/OFF valve 17, and through line 24 to a automated ON/OFF value 13. The water then flows through line 23 to the Mixer 12. A mixer, for example, may be a commercial mixer such as “Quick Mix”.

Control valve 13 is turned on and off by motion detector 14. In this manner, when a vehicle approaches the disinfectant station, control valve 13 is turned on allowing water to flow through mixer 12. As the water flows through mixer 12 it draws and mixes disinfectant with the water before it is sent to the spray nozzles. The motion detector 14 detects vehicles approaching the station from each direction. Alternately, two motion detectors could be used, one for incoming vehicles and one for out going vehicles.

A light 16 may be used to illuminate the station at night. A light 16 is turned on by the motion detector 14 as the vehicle approaches the station, and turned off after the vehicle has left the area, and no more motion is detected. A light control unit 15 is placed in between the motion detector 14 and light 16 so that the light would not be turned on during daylight hours.

Power to the motion detector 14 and light control 15 is supplied through a power line 20, and may be disabled by a switch 25.

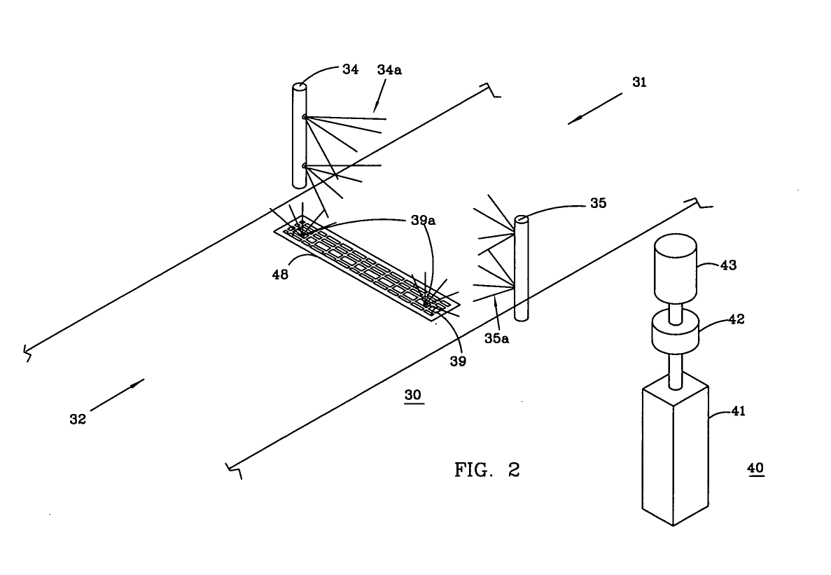

FIG. 2 shows an example of the drive through disinfectant station 30. The vehicle may enter from either end 31 or 32. As the vehicle approaches the station, the motion detector 42 detects the vehicle and turns on the spray station in 41, in the control station 40. The disinfectant is spray through sprayers 34 and 35 Each sprayer 34 and 35 has, respectively, at least one spray nozzle 34a-35a which are directed against the wheels and the sides of the vehicle. Another sprayer 39, having at least two nozzles 39a, are in an opening over which the vehicle passes. A grate 48 covers sprayer 39. Nozzles 34a-35a and 39a may be adjustable to alter the spray patterns to ensure complete coverage of both large and small vehicles. Sprayer 39 directs disinfectant onto the inside surfaces of the wheels. With additional nozzles 39a, the underside of the vehicle may be sprayed. Additional nozzles 39a may be alternately angled so that some spray in one direction and the others in another directions so that both the front and rear surfaces of the vehicle are sprayed as the vehicle moves over sprayer 39.

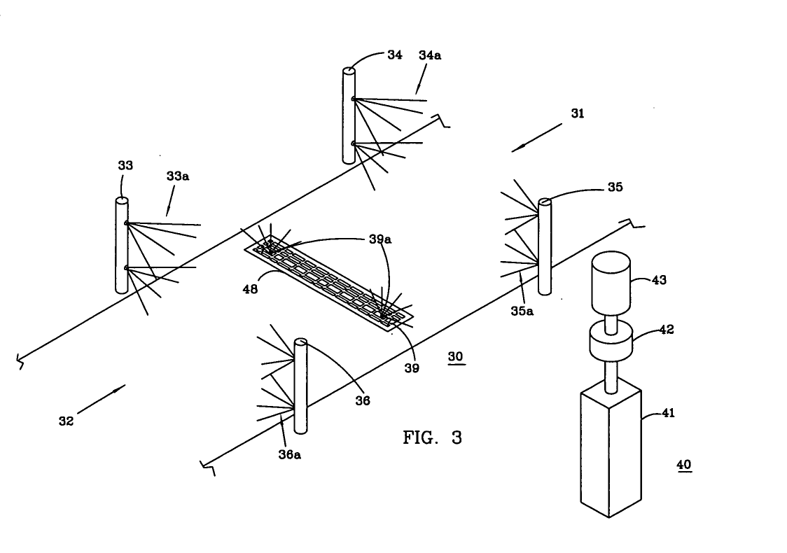

FIG. 3 shows an example of the drive through disinfectant station 30 with four sprayers in stead of two as shown in FIG. 2. The vehicle may enter from either end 31 or 32. As the vehicle approaches the station, the motion detector 42 detects the vehicle and turns on the spray station in 41, in the control station 40. The disinfectant is spray through sprayers 33, 34, 35 and 36. Each sprayer 33-36 has, respectively, at least one spray nozzle 33a-36a which are directed against the wheels and the sides of the vehicle. Another sprayer 39, having at least two nozzles 39a, are in an opening over which the vehicle passes. A grate 48 covers sprayer 39. Nozzles 33a-36a and 39a may be adjustable to alter the spray patterns to ensure complete coverage of both large and small vehicles. Sprayer 39 directs disinfectant onto the inside surfaces of the wheels. With additional nozzles 39a, the underside of the vehicle may be sprayed. Additional nozzles 39a may be alternately angled so that some spray in one direction and the others in another directions so that both the front and rear surfaces of the vehicle are sprayed as the vehicle moves over sprayer 39.

In an alternate embodiment, there may be two under vehicle sprayers (not illustrated), one on each end of the spray area. Each would be tilted inward toward the middle of the spray area. In this embodiment, the front and under side of the vehicle would be sprayed upon the vehicle entering the spray area, and the under side and rear of the vehicle would be sprayed as the vehicle exited the spray area.

What is claimed:

1. An automated disinfectant station for vehicles, comprising;
a plurality of spray nozzles;
a control station for mixing water with a disinfectant liquid; and
a control means for automatically initiating disinfectant spray only while the vehicle is in a location adjacent the spray nozzles.

2. The automated disinfectant station according to claim 1, wherein spraying is initiated and terminated by a motion detector.

3. The automated disinfectant station according to claim 1, wherein the spray station may be entered and activated from two different directions.

4. The automated disinfectant station according to claim 1, including a lighting system automatically activated when a vehicle approaches the disinfectant station from two directions.

5. The automated disinfectant station according to claim 4, wherein the lighting system is automatically disable during day light hours.

6. The automated disinfectant station according to claim 1, wherein the control station for mixing water with a disinfectant liquid includes a mixing value in which the proportions of water and disinfectant may be varied.

7. The automated disinfectant station according to claim 1, wherein water introduced into a mixer for mixing water and disinfectant is controlled by a valve controlled by a motion detector.

8. The automated disinfectant station according to claim 1, wherein the plurality of spray nozzles are positioned to spray sides of the vehicle wheels, vehicle underside, and lower regions of the vehicle.

9. The automated disinfectant station according to claim 1, wherein spraying pressure of the nozzles is adjustable by controlling the water pressure to the nozzles.

10. An automated disinfectant station for vehicles, comprising;
a plurality of spray nozzles;
a control station for mixing water with a disinfectant liquid; and
a motion detector for automatically initiating disinfectant spray only while the vehicle is in a location adjacent the spray nozzles.

11. The automated disinfectant station according to claim 10, wherein the spray station may be entered and activated from two different directions.

12. The automated disinfectant station according to claim 10, including a lighting system automatically activated when a vehicle approaches the disinfectant station from two directions.

13. The automated disinfectant station according to claim 12, wherein the lighting system is automatically disable during day light hours.

14. The automated disinfectant station according to claim 10, wherein the control station for mixing water with a disinfectant liquid includes a mixing value in which the proportions of water and disinfectant may be varied.

15. The automated disinfectant station according to claim 10, wherein water introduced into a mixer for mixing water and disinfectant is controlled by a valve controlled by the motion detector.

16. The automated disinfectant station according to claim 10, wherein the plurality of spray nozzles are positioned to spray sides of the vehicle wheels, vehicle underside, and lower regions of the vehicle.

17. The automated disinfectant station according to claim 10, wherein spraying pressure of the nozzles is adjustable by controlling the water pressure to the nozzles.

18. An automated disinfectant station for vehicles, comprising;
a spray station which may be entered from two different directions;
a plurality of spray nozzles;
a control station for mixing water with a disinfectant liquid; and
a motion detector for automatically initiating disinfectant spray only while the vehicle is in a location adjacent the spray nozzles.

19. The automated disinfectant station according to claim 18, wherein the control station for mixing water with a disinfectant liquid includes a mixing value in which the proportions of water and disinfectant may be varied.

20. The automated disinfectant station according to claim 18, wherein water introduced into a mixer for mixing water and disinfectant is controlled by a valve controlled by the motion detector
